(19) United States
(12) Patent Application Publication
(10) Pub. No.: US 2025/0093291 A1
(43) Pub. Date: Mar. 20, 2025

(54) NUCLEIC ACID PROBE, CHEMICAL SENSOR, AND DETECTION METHOD

(71) Applicant: KABUSHIKI KAISHA TOSHIBA, Tokyo (JP)

(72) Inventors: Yoshiaki SUGIZAKI, Fujisawa Kanagawa (JP); Hiroko MIKI, Kawasaki Kanagawa (JP)

(73) Assignee: KABUSHIKI KAISHA TOSHIBA, Tokyo (JP)

(21) Appl. No.: 18/760,449

(22) Filed: Jul. 1, 2024

(30) Foreign Application Priority Data
Sep. 19, 2023 (JP) 2023-151450

(51) Int. Cl. G01N 27/327; C12N 15/115; G01N 33/94

(52) U.S. Cl. CPC G01N 27/3276; C12N 15/115; G01N 33/946; C12N 2310/16

(57) ABSTRACT

A nucleic acid probe includes first and second nucleic acids. The first nucleic acid includes a binding site. The first nucleic acid includes first to seventh base sequences. The fourth base sequence is complimentary with the third base sequence. The fifth base sequence is complimentary with the second base sequence. The seventh base sequence is complimentary with the sixth base sequence. The binding site is positioned at least between the fourth base sequence and the seventh base sequence. The second nucleic acid includes an eighth base sequence. The eighth base sequence is complimentary with at least a portion of the first base sequence and at least a portion of the second base sequence. A number of bases of the eighth base sequence is more than a number of bases of the second base sequence.

### CROSS-REFERENCE TO RELATED APPLICATIONS

This application is based upon and claims the benefit of priority from Japanese Patent Application No. 2023-151450, filed on Sep. 19, 2023; the entire contents of which are incorporated herein by reference.

### REFERENCE TO A SEQUENCE LISTING

In accordance with 37 CFR § 1.831, the present specification makes reference to a Sequence Listing submitted electronically as a .xml file named “202736US. sequence xml”. The .xml file was generated on “Sep. 17, 2024” and is “8,103” bytes in size. The entire contents of the Sequence Listing are hereby incorporated by reference.

### FIELD

Embodiments described herein relate generally to a nucleic acid probe, a chemical sensor, and a detection method.

### BACKGROUND

For example, a cocaine aptamer that can detect cocaine is proposed in Patent Literature 1.

### BRIEF DESCRIPTION OF THE DRAWINGS

FIG. 1 is a schematic view illustrating a chemical sensor according to an embodiment;

FIG. 2 is a schematic view illustrating the nucleic acid probe according to the embodiment;

FIG. 3 is a schematic view illustrating a structure of the nucleic acid probe according to the embodiment in a first state;

FIG. 4 is a schematic view illustrating a structure of the nucleic acid probe according to the embodiment in a state between the first state and a second state;

FIG. 5 is a schematic view illustrating a structure of the nucleic acid probe according to the embodiment in the second state;

FIGS. 6A to 6D are schematic views illustrating a detection method according to an embodiment;

FIG. 7 is a table illustrating simulation results of reference examples 1 to 8; and

FIG. 8 is a table illustrating simulation results of reference examples 9 to 13.

### DETAILED DESCRIPTION

According to an embodiment of the invention, a nucleic acid probe includes a first nucleic acid and a second nucleic acid. The first nucleic acid includes a binding site configured to trap a target substance. The second nucleic acid is configured to pair with a portion of the first nucleic acid. The first nucleic acid includes a first base sequence, a second base sequence, a third base sequence, a fourth base sequence, a fifth base sequence, a sixth base sequence, and a seventh base sequence. The second base sequence is bound to the first base sequence. The third base sequence is bound to the second base sequence. The fourth base sequence is complimentary with the third base sequence. The fifth base sequence is complimentary with the second base sequence. The sixth base sequence is bound to the fifth base sequence. The seventh base sequence is complimentary with the sixth base sequence. The binding site is positioned at least between the fourth base sequence and the seventh base sequence. The second nucleic acid includes an eighth base sequence. The eighth base sequence is complimentary with at least a portion of the first base sequence and at least a portion of the second base sequence. A number of bases of the eighth base sequence is more than a number of bases of the second base sequence.

Various embodiments will be described hereinafter with reference to the accompanying drawings.

The drawings are schematic and conceptual; and the relationships between the thickness and width of portions, the proportions of sizes among portions, etc., are not necessarily the same as the actual values thereof. Further, the dimensions and proportions may be illustrated differently among drawings, even for identical portions.

In the specification and drawings, components similar to those described or illustrated in a drawing thereinabove are marked with like reference numerals, and a detailed description is omitted as appropriate.

### Chemical Sensor

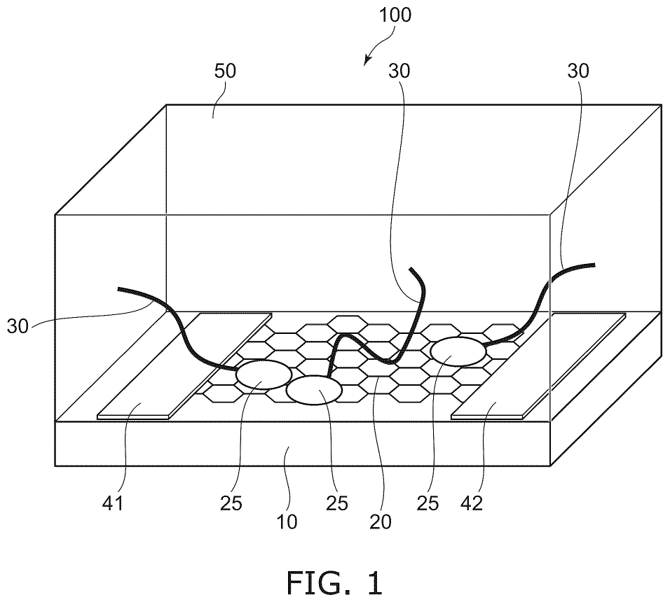

FIG. 1 is a schematic view illustrating a chemical sensor according to an embodiment.

As illustrated in FIG. 1, the chemical sensor 100 according to the embodiment includes a substrate 10, a sensor element 20, a nucleic acid probe 30, a first electrode 41, and a second electrode 42.

The sensor element 20, the first electrode 41, and the second electrode 42 each are located on the substrate 10. The first electrode 41 and the second electrode 42 each are electrically connected with the sensor element 20.

For example, the chemical sensor 100 has a FET (field effect transistor) structure. One of the first electrode 41 or the second electrode 42 functions as a drain electrode. The other of the first electrode 41 or the second electrode 42 functions as a source electrode. A current flows between the first electrode 41 and the second electrode 42 via the sensor element 20.

The sensor element 20 is, for example, graphene. The substrate 10 is, for example, a silicon substrate. For example, the graphene can be located on the substrate 10 with a foundation film (not illustrated) interposed. For example, a silicon oxide film can be used as the foundation film. Also, the foundation film can be provided with the function of a chemical catalyst for forming the graphene. The sensor element 20 may be, for example, an ion-sensitive field-effect transistor (ISFET).

The nucleic acid probe 30 specifically recognizes a target substance 1. More specifically, the nucleic acid probe 30 includes a binding site (a binding site 31s described below) that is configured to trap the target substance 1. When the binding site traps the target substance 1, the target substance 1 can be detected by a change of the structure of the nucleic acid probe 30. The nucleic acid probe 30 may be configured to recognize not only the target substance 1, but also a substance having a similar structure such as a derivative of the target substance 1, etc. The structure of the nucleic acid probe 30 is described below.

The nucleic acid probe 30 is positioned at the surface of the sensor element 20. The nucleic acid probe 30 is immobilized at the surface of the sensor element 20 via, for example, a linker (or scaffold molecule) 25. The nucleic acid probe 30 is bound to the linker 25. For example, pyrene can be used as the linker 25. The nucleic acid probe 30 being positioned at the surface of the sensor element 20 refers to the nucleic acid probe 30 being immobilized at the surface of the sensor element 20 by the nucleic acid probe 30 being bound to, adsorbed to, or approaching the surface of the sensor element 20 due to a chemical or charge-induced attraction, hydrophobic interaction, etc.

The nucleic acid probe 30 and the surface of the sensor element 20 are immersed in a solution 50. The solution 50 is, for example, an aqueous solution. The solution 50 is, for example, a buffer solution. Or, a water-soluble organic solvent DMSO sulfoxide), such as (dimethyl DMF (N,N-dimethylformamide), a lower alcohol, etc., may be used as a major component.

The first electrode 41 and the second electrode 42 are covered with a protective film (not illustrated). The protective film suppresses direct contact of the first and second electrodes 41 and 42 with the nucleic acid probe 30, the target substance 1, or the solution 50. For example, the graphene surface may be covered with an insulating body such as a phospholipid film, etc., as necessary.

When the nucleic acid probe 30 traps the target substance 1, the electronic state of the sensor element 20 changes due to a structural change of the nucleic acid probe 30 caused by the trapping of the target substance 1. The change of the electronic state is detected as a change of the current flowing between the first electrode 41 and the second electrode 42, by which the presence of the target substance 1 or the concentration of the target substance 1 in a specimen can be detected.

### Nucleic Acid Probe

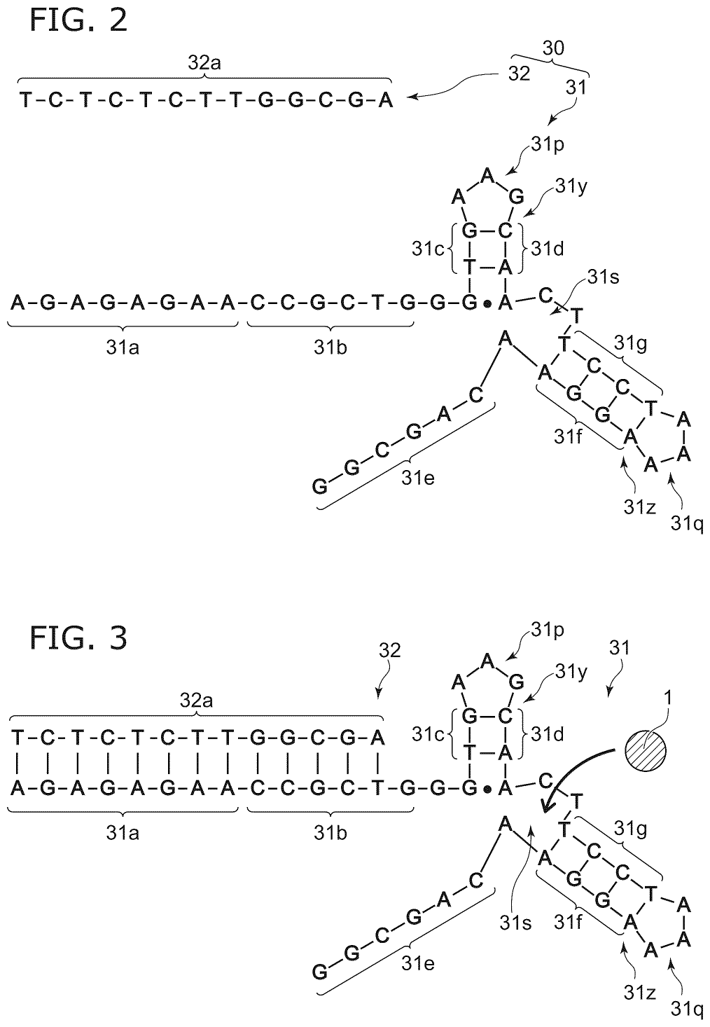

FIG. 2 is a schematic view illustrating the nucleic acid probe according to the embodiment.

As illustrated in FIG. 2, the nucleic acid probe 30 includes a first nucleic acid 31 and a second nucleic acid 32. The first nucleic acid 31 includes the binding site 31s that is configured to trap the target substance 1. The second nucleic acid 32 can pair with a portion of the first nucleic acid 31.

The first nucleic acid 31 and the second nucleic acid 32 are, for example, DNA (deoxyribonucleic acid). In the drawings, “A” means adenine, “T” means thymine, “G” means guanine, and “C” means cytosine. The first nucleic acid 31 and the second nucleic acid 32 may be RNA (ribonucleic acid). In such a case, uracil is included instead of thymine.

The first nucleic acid 31 is, for example, a cocaine aptamer. The cocaine aptamer is, for example, an aptamer that can specifically recognize a cocaine molecule. The cocaine aptamer is, for example, an aptamer that can specifically interact with a cocaine molecule. When the first nucleic acid 31 is a cocaine aptamer, the target substance 1 is a cocaine molecule, or a methyl benzoate molecule having a structure similar to that of a cocaine molecule.

The first nucleic acid 31 includes first to seventh base sequences 31a to 31g. The number of bases is, for example, not more than 20 for each of the first to seventh base sequences 31a to 31g. For example, the first base sequence 31a is positioned at the 3′ end or 5′ end of the first nucleic acid 31. Although the first base sequence 31a is at the 3′ end of the first nucleic acid 31 in an example described hereinbelow, the first base sequence 31a position may be the opposite. When describing the base sequences hereinbelow, the base sequences are described in order from the 5′ side toward the 3′ side. In the example, the portion “AAGAGAGA” corresponds to the first base sequence 31a. The number of bases of the first base sequence 31a is not less than 2. The number of bases of the first base sequence 31a may be, for example, 6 to 8. When the number of bases of the first base sequence 31a is not less than 4, it is favorable for the first base sequence 31a not to be complimentary with the other base sequences of the first nucleic acid 31. The first nucleic acid 31 has a single strand in the example described hereinbelow. The first nucleic acid 31 may include multiple strands; the first to third base sequences 31a to 31c may be included in the same strand (a first strand); the fourth and seventh base sequences 31d and 31g may be included in the same strand (a second strand); and the fifth and sixth base sequences 31e and 31f may be included in the same strand (a third strand).

The second base sequence 31b is bound to the 5′ end of the first base sequence 31a. The second base sequence 31b is continuous with the first base sequence 31a. In the example, the portion “GTCGCC” corresponds to the second base sequence 31b. The number of bases of the second base sequence 31b is not less than 2. Favorably, the number of bases of the second base sequence 31b is not less than 4.

The third base sequence 31c is bound to the 5′ end of the second base sequence 31b via a spacer sequence (GG). The second base sequence 31b is positioned between the first base sequence 31a and the third base sequence 31c. In the example, the portion “GT” corresponds to the third base sequence 31c. The number of bases of the third base sequence 31c is not less than 2.

The fourth base sequence 31d is complimentary with the third base sequence 31c. In the example, the fourth base sequence 31d is bound to the 5′ end of the third base sequence 31c via a first loop portion 31p. In the example, the portion “GAA” corresponds to the first loop portion 31p. In the example, the portion “AC” corresponds to the fourth base sequence 31d. The number of bases of the fourth base sequence 31d is not less than 2. The first loop portion 31p may not be included. The third base sequence 31c and the fourth base sequence 31d may be separate strands.

The fifth base sequence 31e is complimentary with the second base sequence 31b. For example, the fifth base sequence 31e is positioned at the end of the first nucleic acid 31 at the side opposite to the first base sequence 31a. In the example, the fifth base sequence 31e is positioned at the 5′ end of the first nucleic acid 31. Another base sequence may be bound to the 5′ end of the fifth base sequence 31e. In the example, the portion “GGCGAC” corresponds to the fifth base sequence 31e. The number of bases of the fifth base sequence 31e is not less than 2.

The sixth base sequence 31f is bound to the fifth base sequence 31e via a spacer sequence (A). In the example, the portion of “AGGA” corresponds to the sixth base sequence 31f. The number of bases of the sixth base sequence 31f is not less than 2.

The seventh base sequence 31g is complimentary with the sixth base sequence 31f. In the example, the seventh base sequence 31g is bound to the 3′ end of the sixth base sequence 31f via a second loop portion 31q. In the example, the portion “AAA” corresponds to the second loop portion 31q. In the example, the portion “TCCT” corresponds to the seventh base sequence 31g. The number of bases of the seventh base sequence 31g is not less than 2. The second loop portion 31q may not be included. The sixth base sequence 31f and the seventh base sequence 31g may be separate strands.

The binding site 31s is present between stems of the first nucleic acid 31. The binding site 31s is formed of at least one base between the second base sequence 31b and the third base sequence 31c, at least one base between the fourth base sequence 31d and the seventh base sequence 31g, and at least one base between the fifth base sequence 31e and the sixth base sequence 31f. In the example, the portion “TCA” corresponds to the binding site 31s.

The second nucleic acid 32 includes an eighth base sequence 32a. The number of bases of the eighth base sequence 32a is, for example, not more than 20. The number of bases of the second base sequence 32a may be, for example, 10 to 13. The eighth base sequence 32a is complimentary with at least a portion of the first base sequence 31a and at least a portion of the second base sequence 31b. In the example, the eighth base sequence 32a is complimentary with the entire first base sequence 31a and a portion of the second base sequence 31b. In the example, the portion “TCTCTCTTGGCGA” (the entire second nucleic acid 32) corresponds to the eighth base sequence 32a. Among the portion “TCTCTCTTGGCGA”, the portion “GGCGA” is complimentary with the second base sequence 31b; and the portion “TCTCTCTT” is complimentary with the first base sequence 31a. The second nucleic acid 32 may include another base sequence bound to the 3′ end of the eighth base sequence 32a, and may include another base sequence bound to the 5′ end of the eighth base sequence 32a.

The number of bases of the eighth base sequence 32a is more than the number of bases of the second base sequence 31b and the number of bases of the fifth base sequence 31e. The number of bases of the eighth base sequence 32a is not less than 4. It is favorable for the number of bases of the eighth base sequence 32a to be not less than 10. The number of bases of the portion of the eighth base sequence 32a that is complimentary with the first base sequence 31a is not less than 2. The number of bases of the portion of the eighth base sequence 32a that is complimentary with the second base sequence 31b is not less than 2. It is desirable for the number of bases of the portion of the eighth base sequence 32a that is complimentary with the second base sequence 31b to be not less than half of the number of bases of the second base sequence 31b. The number of bases of the portion of the eighth base sequence 32a that is complimentary with the first base sequence 31a may be more than the number of bases of the portion of the eighth base sequence 32a that is complimentary with the second base sequence 31b, may be less than the number of bases of the portion of the eighth base sequence 32a that is complimentary with the second base sequence 31b, or may be equal to the number of bases of the portion of the eighth base sequence 32a that is complimentary with the second base sequence 31b.

Spacer sequences may be provided between the base sequences. The numbers of bases included in the spacer sequences are, for example, not less than 1 and not more than 3.

FIG. 3 is a schematic view illustrating a structure of the nucleic acid probe according to the embodiment in a first state.

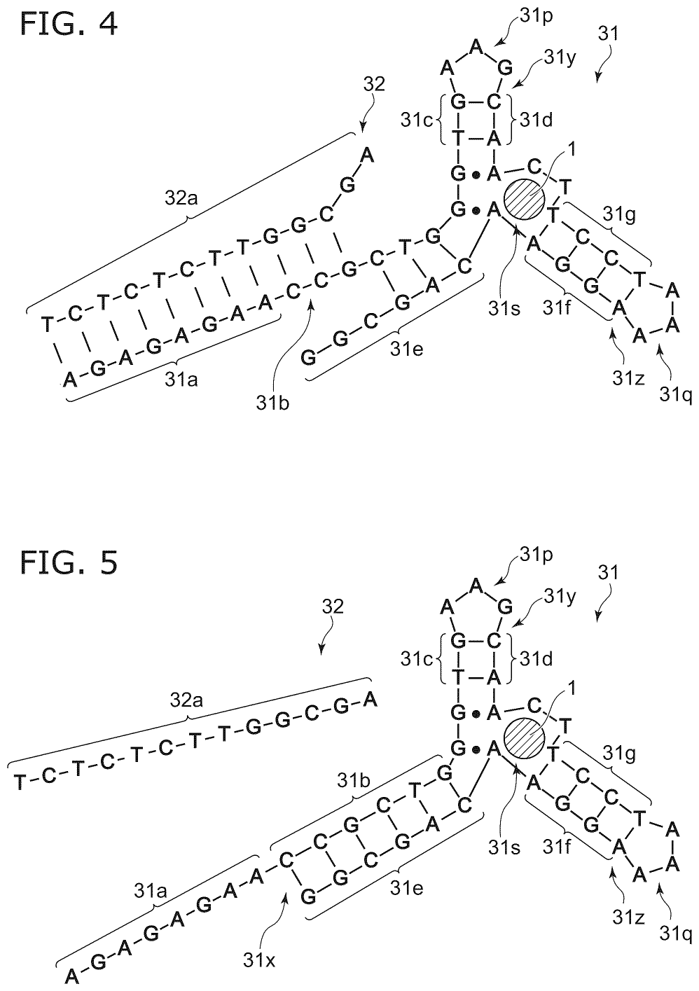

FIG. 4 is a schematic view illustrating a structure of the nucleic acid probe according to the embodiment in a state between the first state and a second state.

FIG. 5 is a schematic view illustrating a structure of the nucleic acid probe according to the embodiment in the second state.

As illustrated in FIGS. 3 to 5, the nucleic acid probe 30 includes the first and second states.

As illustrated in FIG. 3, the first state is a state in which the second nucleic acid 32 is paired with the first nucleic acid 31. As illustrated in FIG. 5, the second state is a state in which second nucleic acid 32 is dissociated from the first nucleic acid 31.

In the second state, the first nucleic acid 31 forms a high-order structure including a first stem portion 31x, a second stem portion 31y, and a third stem portion 31z. The first stem portion 31x is formed by the pairing of the second and fifth base sequences 31b and 31e. The second stem portion 31y is formed by the pairing of the third and fourth base sequences 31c and 31d. For example, the second stem portion 31y forms a stem-loop structure together with the first loop portion 31p. The third stem portion 31z is formed by the pairing of the sixth and seventh base sequences 31f and 31g. For example, the third stem portion 31z forms a stem-loop structure together with the second loop portion 31q.

In the first state, the first nucleic acid 31 forms a structure that does not include at least the first stem portion 31x. In the example, in the first state, the first nucleic acid 31 forms a structure that includes the second and third stem portions 31y and 31z but does not include the first stem portion 31x. In the first state, the first nucleic acid 31 may not include the second stem portion 31y and the third stem portion 31z. In the first state, the second nucleic acid 32 inhibits the formation of the first stem portion 31x by pairing with the second base sequence 31b of the first nucleic acid 31.

The nucleic acid probe 30 enters the second state when the binding site 31s traps the target substance 1 in the first state. More specifically, when the binding site 31s traps the target substance 1 in the first state as illustrated in FIGS. 3 and 4, the second and fifth base sequences 31b and 31e of the first nucleic acid 31 become paired; and the eighth base sequence 32a of the second nucleic acid 32 and the second base sequence 31b of the first nucleic acid 31 become unpaired. When the second and fifth base sequences 31b and 31e of the first nucleic acid 31 are completely paired as illustrated in FIG. 5, the eighth base sequence 32a of the second nucleic acid 32 and the first base sequence 31a of the first nucleic acid 31 then become unpaired, and the second nucleic acid 32 dissociates from the first nucleic acid 31.

For example, when the nucleic acid probe 30 enters the second state, the second state is maintained even when the target substance 1 is not trapped by the binding site 31s. For example, the nucleic acid probe 30 enters the first state when the concentration of the second nucleic acid 32 around the first nucleic acid 31 in the second state exceeds a prescribed value. As a result, for example, after the target substance 1 is detected (in the second state), a solution that includes a high concentration of the second nucleic acid 32 can be caused to contact the nucleic acid probe 30, and then the concentration of the second nucleic acid 32 can be reduced or the excess second nucleic acid 32 can be removed by washing to return the nucleic acid probe 30 to the first state. Thus, the nucleic acid probe 30 can be repeatedly used by reversibly changing the state of the nucleic acid probe 30 between the first state and the second state.

In the example, the high-order structure is a three-way junction (3-way junction). The high-order structure may be a four-way junction (4-way junction). That is, in the second state, the first nucleic acid 31 may form a high-order structure that includes another stem portion in addition to the first to third stem portions 31x to 31z.

It is favorable for the melting temperature (Tm) of the first nucleic acid 31 in the second state to be not less than 25° C. As long as the melting temperature (Tm) of the first nucleic acid 31 in the second state is not less than 25° C., the nucleic acid probe 30 can be set to the second state at room temperature (the temperature at which the target substance 1 is detected). That is, the nucleic acid probe 30 is easily used at room temperature (the temperature at which the target substance 1 is detected). In the example, the melting temperature of the first nucleic acid 31 in the second state is 52.3° C. For example, the melting temperature can be calculated using the Oligo Analyzer Tool, which is a calculation tool published by the website of Integrated DNA Technologies, Inc. Or, experimental measurement can be performed using UV absorption measurement, circular dichroism measurement, differential calorimetry, etc.

As described above, the nucleic acid probe 30 is immobilized at the surface of the sensor element 20. Either the first nucleic acid 31 or the second nucleic acid 32 may be immobilized at the surface of the sensor element 20. When the first nucleic acid 31 is immobilized at the surface of the sensor element 20, the 3′ end of the first nucleic acid 31 may be immobilized at the surface of the sensor element 20, or the 5′ end of the first nucleic acid 31 may be immobilized at the surface of the sensor element 20. When the second nucleic acid 32 is immobilized at the surface of the sensor element 20, the 3′ end of the second nucleic acid 32 may be immobilized at the surface of the sensor element 20, or the 5′ end of the second nucleic acid 32 may be immobilized at the surface of the sensor element 20.

To detect the target substance with high sensitivity, it is favorable for the nucleic acid probe 30 to be able to stably maintain the first state (the state in which the target substance has not been trapped) until the binding site 31s traps the target substance 1. However, according to the structures of the first and second nucleic acids 31 and 32, there are cases where the first state cannot be stably maintained until the binding site 31s traps the target substance 1.

Therefore, according to the embodiment, the number of bases of the eighth base sequence 32a, which is the portion of the second nucleic acid 32 paired with the first nucleic acid 31, is set to be more than the number of bases of the second base sequence 31b, which is the portion of the first nucleic acid 31 forming the first stem portion 31x. As a result, the number of base pairs that are paired between the first nucleic acid 31 and the second nucleic acid 32 can be greater than the number of base pairs that are paired between the second base sequence 31b and the fifth base sequence 31e. Accordingly, the first state in which the second nucleic acid 32 is paired with the first nucleic acid 31 can be stably maintained until the binding site 31s traps the target substance 1. As a result, the target substance 1 can be detected with high sensitivity.

### Detection Method

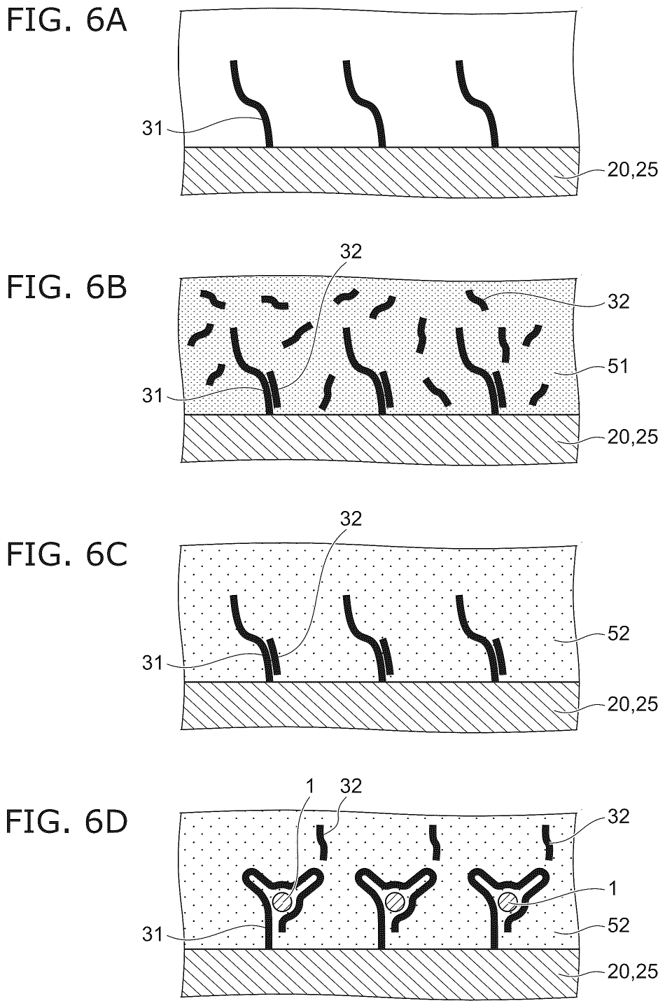

FIGS. 6A to 6D are schematic views illustrating a detection method according to an embodiment.

As illustrated in FIGS. 6A to 6D, the detection method according to the embodiment is a detection method of the target substance 1 that uses the nucleic acid probe 30 according to the embodiment above. The detection method according to the embodiment includes first to fourth processes.

FIG. 6A illustrates the first process. The first process is a process of immobilizing one of the first nucleic acid 31 or the second nucleic acid 32 at the surface of the sensor element 20. In the first process of the example, the first nucleic acid 31 is immobilized at the surface of the sensor element 20. For example, one of the first nucleic acid 31 or the second nucleic acid 32 can be immobilized at the surface of the sensor element 20 by causing a reagent (a solution) including the one of the first nucleic acid 31 or the second nucleic acid 32 to contact the surface of the sensor element 20. The excess reagent can be removed by washing.

FIG. 6B illustrates the second process. The second process is performed after the first process. The second process is a process of pairing the other of the first nucleic acid 31 or the second nucleic acid 32 with the one of the first nucleic acid 31 or the second nucleic acid 32 immobilized at the surface of the sensor element 20 by causing a first solution 51, in which the concentration of the other of the first nucleic acid 31 or the second nucleic acid 32 in the first solution 51 is a first concentration, to contact the surface of the sensor element 20. In the second process of the example, the second nucleic acid 32 is paired with the first nucleic acid 31 immobilized at the surface of the sensor element 20 by causing the first solution 51, in which the concentration of the second nucleic acid 32 is the first concentration, to contact the surface of the sensor element 20. The first concentration is, for example, not less than 100 nM, and more desirably not less than 1 μM.

FIG. 6C illustrates the third process. The third process is performed after the second process. The third process is a process of replacing the first solution 51 at the surface of the sensor element 20 with a second solution 52, in which the concentration of the other of the first nucleic acid 31 or the second nucleic acid 32 is a second concentration that is less than the first concentration. In the third process of the example, the first solution 51 at the surface of the sensor element 20 is replaced with the second solution 52, in which the concentration of the free second nucleic acid 32 is the second concentration that is less than the first concentration. The second concentration is, for example, not less than 100 fM and not more than 10 nM. Or, the second solution 52 may not include free second nucleic acid 32.

FIG. 6D illustrates the fourth process. The fourth process is performed after the third process. The fourth process is a process of detecting the target substance 1 in a state in which the surface of the sensor element 20 is covered with the second solution 52. A solution that includes the specimen is introduced and caused to contact the sensor element 20, or a gas that includes the specimen is brought into contact with the second solution 52 and dissolved. When the target substance 1 is included in the specimen, the state of the first and second nucleic acids 31 and 32 is irreversibly changed at the vicinity of the sensor element 20. The target substance 1 can be detected with high sensitivity by measuring the change of the electrical characteristics between the first electrode 41 and the second electrode 42.

According to such a method, the nucleic acid probe 30 can be formed at the surface of the sensor element 20 by using the reagent (the solution) including the first nucleic acid 31 and the reagent (the solution) including the second nucleic acid 32. Also, by detecting the target substance 1 in a state in which the surface of the sensor element 20 is covered with the second solution 52, the first nucleic acid 31 easily changes from the first state to the second state when the target substance 1 is trapped.

In the example, the first nucleic acid 31 is immobilized at the surface of the sensor element 20 in the first process; and the second nucleic acid 32 is paired with the first nucleic acid 31 immobilized at the surface of the sensor element 20 in the second process. The second nucleic acid 32 may be immobilized at the surface of the sensor element 20 in the first process; and the first nucleic acid 31 may be paired with the second nucleic acid 32 immobilized at the surface of the sensor element 20 in the second process.

### Reference Examples

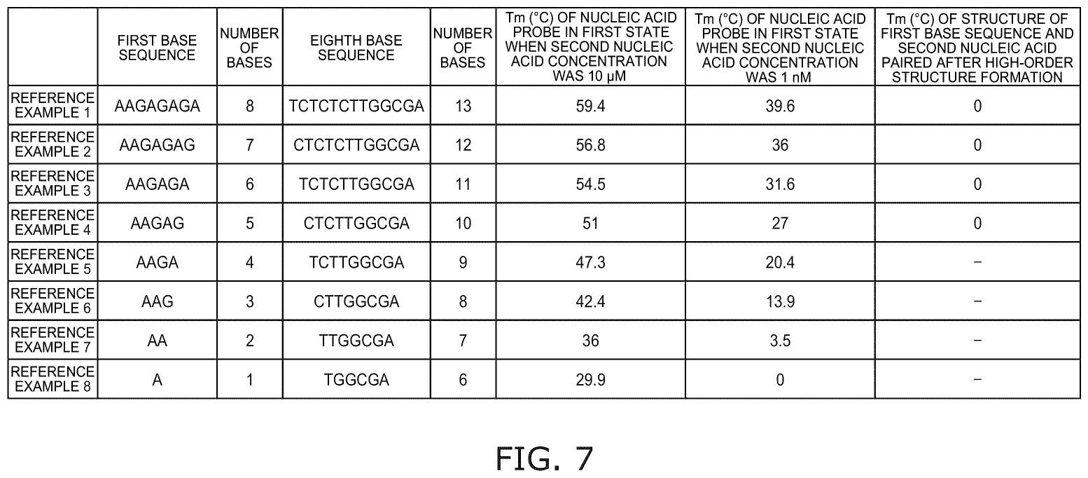

FIG. 7 is a table illustrating simulation results of reference examples 1 to 8.

For the reference examples 1 to 8, the first base sequence 31a of the first nucleic acid 31 illustrated in FIG. 2 was assumed to be as shown in FIG. 7; and the eighth base sequence 32a of the second nucleic acid 32 illustrated in FIG. 2 was assumed to be as shown in FIG. 7. For the reference examples 1 to 8, the results calculated by simulation for the melting temperature (Tm) of the nucleic acid probe 30 in the first state when the concentration of the second nucleic acid 32 was 10 μM, the melting temperature (Tm) of the nucleic acid probe 30 in the first state when the concentration of the second nucleic acid 32 was 1 nM, and the melting temperature (Tm) of the structure in which the first base sequence 31a and the second nucleic acid 32 (the eighth base sequence 32a) were paired after the high-order structure formation are shown in FIG. 7. The melting temperature (Tm) of the first nucleic acid 31 in the second state was 52.3° C.

As illustrated in FIG. 7, compared to when the number of bases of the eighth base sequence 32a was low, increasing the number of bases of the eighth base sequence 32a resulted in higher melting temperatures (Tm) of the nucleic acid probe 30 in the first state when the concentration of the second nucleic acid 32 was 10 μM, and resulted in higher melting temperatures (Tm) of the nucleic acid probe 30 in the first state when the concentration of the second nucleic acid 32 was 1 nM. Therefore, this suggests that increasing the number of bases of the eighth base sequence 32a makes it possible for the nucleic acid probe 30 to more stably maintain the first state (that is, the state in which the target substance 1 had not been trapped) than when the number of bases of the eighth base sequence 32a is low.

More specifically, in the reference examples 1 to 3 as illustrated in FIG. 7, when the concentration of the second nucleic acid 32 was 10 μM, the melting temperature (Tm) of the nucleic acid probe 30 in the first state was greater than the melting temperature of the first nucleic acid 31 in the second state. Therefore, this suggests that the nucleic acid probe 30 could more stably maintain the first state (that is, the state in which the target substance 1 had not been trapped) in the reference examples 1 to 3 than in the reference examples 4 to 8. Also, in the reference examples 1 to 3, the melting temperature (Tm) of the nucleic acid probe 30 in the first state when the concentration of the second nucleic acid 32 was 1 nM was not less than 30° C. and not less than room temperature. Therefore, this suggests that in the reference examples 1 to 3, the nucleic acid probe 30 could more stably maintain the first state (that is, the state in which the target substance 1 had not been trapped) even when the concentration of the second nucleic acid 32 was 1 nM. When, however, there is competition in the high-order structure formation of the first nucleic acid 31, conditions may occur in which the first state cannot be stably maintained due to the occurrence of a strand exchange reaction. When the seventh “A” and the twenty-first “A” from the 540 side approach each other due to the target substance 1 binding to the first nucleic acid 31, the two Gs forming the spacer sequence between the third base sequence 31c and the second base sequence 31b form G-A mismatches with these two As. As a result, as shown in FIG. 4, when the second stem portion 31y side of the first stem portion 31x starts to continuously form from the second stem portion 31y via the two G-A mismatches, the first stem portion 31x is formed as-is by the first stem portion 31x replacing the base pairs with the eighth base sequence 32a.

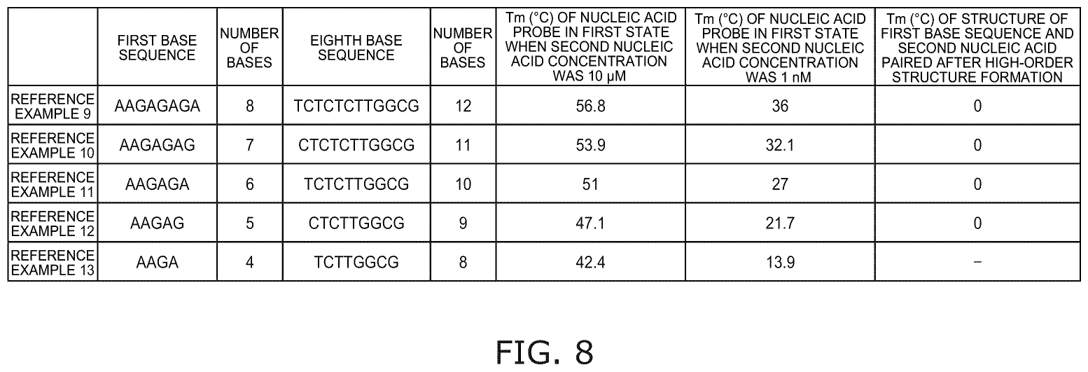

FIG. 8 is a table illustrating simulation results of reference examples 9 to 13.

For the reference examples 9 to 13 as illustrated in FIG. 8, the first base sequence 31a of the first nucleic acid 31 illustrated in FIG. 2 was assumed to be as shown in FIG. 8; and the eighth base sequence 32a of the second nucleic acid 32 illustrated in FIG. 2 was assumed to be as shown in FIG. 8. For the reference examples 9 to 13, results calculated by simulation for the melting temperature (Tm) of the nucleic acid probe 30 in the first state when the concentration of the second nucleic acid 32 was 10 μM, the melting temperature (Tm) of the nucleic acid probe 30 in the first state when the concentration of the second nucleic acid 32 was 1 nM, and the melting temperature (Tm) of the structure in which the first base sequence 31a and the second nucleic acid 32 (the eighth base sequence 32a) were paired after the high-order structure formation are shown in FIG. 8. The melting temperature (Tm) of the first nucleic acid 31 in the second state was 52.3° C.

As illustrated in FIG. 8, compared to when the number of bases of the eighth base sequence 32a was low, increasing the number of bases of the eighth base sequence 32a resulted in higher melting temperatures (Tm) of the nucleic acid probe 30 in the first state when the concentration of the second nucleic acid 32 was 10 μM, and resulted in higher melting temperatures (Tm) of the nucleic acid probe 30 in the first state when the concentration of the second nucleic acid 32 was 1 nM. Therefore, this suggests that increasing the number of bases of the eighth base sequence 32a makes it possible for the nucleic acid probe 30 to more stably maintain the first state (that is, the state in which the target substance 1 had not been trapped) than when the number of bases of the eighth base sequence 32a is low.

More specifically, in the reference examples 9 and 10 as illustrated in FIG. 8, when the concentration of the second nucleic acid 32 was 10 μM, the melting temperature (Tm) of the nucleic acid probe 30 in the first state was greater than the melting temperature of the first nucleic acid 31 in the second state. Therefore, this suggests that the nucleic acid probe 30 could more stably maintain the first state (that is, the state in which the target substance 1 had not been trapped) in the reference examples 9 and 10 than in the reference examples 11 to 13. Also, in the reference examples 9 and 10, the melting temperature (Tm) of the nucleic acid probe 30 in the first state when the concentration of the second nucleic acid 32 was 1 nM was not less than 30° C. Therefore, this suggests that in the reference examples 9 and 10, the nucleic acid probe 30 could stably maintain the first state (that is, the state in which the target substance 1 had not been trapped) when the concentration of the second nucleic acid 32 was 1 nM. When, however, there is competition in the high-order structure formation of the first nucleic acid 31, conditions may occur in which the first state cannot be stably maintained due to the occurrence of a strand exchange reaction. When the seventh “A” and the twenty-first “A” from the 5′ side approach each other due to the target substance 1 binding to the first nucleic acid 31, the two Gs forming the spacer sequence between the third base sequence 31c and the second base sequence 31b form G-A mismatches with these two As. As a result, as shown in FIG. 4, when the second stem portion 31y side of the first stem portion 31x starts to continuously form from the second stem portion 31y via the two G-A mismatches, the first stem portion 31x is formed as-is by the first stem portion 31x replacing the base pairs with the eighth base sequence 32a.

Embodiments may include the following configurations.

### Configuration 1

A nucleic acid probe, comprising:
a first nucleic acid including a binding site, the binding site being configured to trap a target substance; and
a second nucleic acid configured to pair with a portion of the first nucleic acid,
the first nucleic acid including
   a first base sequence,
   a second base sequence bound to the first base sequence,
   a third base sequence bound to the second base sequence,
   a fourth base sequence that is complimentary to the third base sequence,
   a fifth base sequence that is complimentary to the second base sequence,
   a sixth base sequence bound to the fifth base sequence, and
   a seventh base sequence that is complimentary to the sixth base sequence,
the binding site being positioned at least between the fourth base sequence and the seventh base sequence,
the second nucleic acid including an eighth base sequence,
the eighth base sequence being complimentary with at least a portion of the first base sequence and at least a portion of the second base sequence,
a number of bases of the eighth base sequence being more than a number of bases of the second base sequence.

### Configuration 2

The nucleic acid probe according to configuration 1, wherein
the nucleic acid probe includes:
   a first state in which the second nucleic acid is paired with the first nucleic acid; and
   a second state in which
      the second nucleic acid is dissociated from the first nucleic acid, and
      a high-order structure of the first nucleic acid is formed,
the high-order structure includes:
   a first stem portion in which the second base sequence and the fifth base sequence are paired;
   a second stem portion in which the third base sequence and the fourth base sequence are paired; and
   a third stem portion in which the sixth base sequence and the seventh base sequence are paired, and
the first state becomes the second state when the binding site traps the target substance in the first state.

### Configuration 3

The nucleic acid probe according to configuration 2, wherein
a melting temperature of the first nucleic acid in the second state is not less than 25° C.

### Configuration 4

The nucleic acid probe according to any one of configurations 1 to 3, wherein
the first nucleic acid is a cocaine aptamer.

### Configuration 5

The nucleic acid probe according to any one of configurations 1 to 4, wherein
the fourth base sequence is connected to the seventh base sequence via a spacer sequence.

### Configuration 6

The nucleic acid probe according to any one of configurations 1 to 5, wherein
the third base sequence is bound to the fourth base sequence, and
the sixth base sequence is bound to the seventh base sequence.

### Configuration 7

The nucleic acid probe according to any one of configurations 1 to 5, wherein
the first nucleic acid includes:
   a first strand including the first, second, and third base sequences,
   a second strand including the fourth and seventh base sequences, and
   a third strand including the fifth and sixth base sequences.

### Configuration 8

A chemical sensor, comprising:
the nucleic acid probe according to any one of configurations 1 to 7;
a sensor element;
a first electrode electrically connected with the sensor element; and
a second electrode electrically connected with the sensor element,
the nucleic acid probe being positioned at a surface of the sensor element.

### Configuration 9

A detection method,
the method using the nucleic acid probe according to any one of configurations 1 to 7,
the method being a detection method of the target substance,
the method comprising:
   a first process of immobilizing one of the first nucleic acid or the second nucleic acid at a surface of a sensor element;
   a second process of pairing another of the first nucleic acid or the second nucleic acid with the one of the first nucleic acid or the second nucleic acid immobilized at the surface of the sensor element by causing a first solution to contact the surface of the sensor element, a concentration of the other of the first nucleic acid or the second nucleic acid in the first solution being a first concentration;
   a third process of replacing the first solution at the surface of the sensor element with a second solution, a concentration of the other of the first nucleic acid or the second nucleic acid in the second solution being a second concentration, the second concentration being less than the first concentration; and
   a fourth process of detecting the target substance in a state in which the surface of the sensor element is covered with the second solution.

Thus, according to embodiments, a nucleic acid probe, a chemical sensor, and a detection method that can detect a target substance with high sensitivity can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A nucleic acid probe, comprising:
a first nucleic acid including a binding site, the binding site being configured to trap a target substance; and
a second nucleic acid configured to pair with a portion of the first nucleic acid,
the first nucleic acid including
a first base sequence,
a second base sequence bound to the first base sequence,
a third base sequence bound to the second base sequence,
a fourth base sequence that is complimentary to the third base sequence,
a fifth base sequence that is complimentary to the second base sequence,
a sixth base sequence bound to the fifth base sequence, and
a seventh base sequence that is complimentary to the sixth base sequence,
the binding site being positioned at least between the fourth base sequence and the seventh base sequence,
the second nucleic acid including an eighth base sequence,
the eighth base sequence being complimentary with at least a portion of the first base sequence and at least a portion of the second base sequence,
a number of bases of the eighth base sequence being more than a number of bases of the second base sequence.

2. The nucleic acid probe according to claim 1, wherein
the nucleic acid probe includes:
a first state in which the second nucleic acid is paired with the first nucleic acid; and
a second state in which
the second nucleic acid is dissociated from the first nucleic acid, and
a high-order structure of the first nucleic acid is formed,
the high-order structure includes:
a first stem portion in which the second base sequence and the fifth base sequence are paired;
a second stem portion in which the third base sequence and the fourth base sequence are paired; and
a third stem portion in which the sixth base sequence and the seventh base sequence are paired, and
the first state becomes the second state when the binding site traps the target substance in the first state.

3. The nucleic acid probe according to claim 2, wherein
a melting temperature of the first nucleic acid in the second state is not less than 25° C.

4. The nucleic acid probe according to claim 1, wherein
the first nucleic acid is a cocaine aptamer.

5. The nucleic acid probe according to claim 1, wherein
the fourth base sequence is connected to the seventh base sequence via a spacer sequence.

6. The nucleic acid probe according to claim 5, wherein
the third base sequence is bound to the fourth base sequence, and
the sixth base sequence is bound to the seventh base sequence.

7. The nucleic acid probe according to claim 5, wherein
the first nucleic acid includes:
a first strand including the first, second, and third base sequences,
a second strand including the fourth and seventh base sequences, and
a third strand including the fifth and sixth base sequences.

8. A chemical sensor, comprising:
the nucleic acid probe according to claim 1;
a sensor element;
a first electrode electrically connected with the sensor element; and
a second electrode electrically connected with the sensor element,
the nucleic acid probe being positioned at a surface of the sensor element.

9. A detection method,
the method using the nucleic acid probe according to claim 1,
the method being a detection method of the target substance,
the method comprising:
a first process of immobilizing one of the first nucleic acid or the second nucleic acid at a surface of a sensor element;
a second process of pairing another of the first nucleic acid or the second nucleic acid with the one of the first nucleic acid or the second nucleic acid immobilized at the surface of the sensor element by causing a first solution to contact the surface of the sensor element, a concentration of the other of the first nucleic acid or the second nucleic acid in the first solution being a first concentration;
a third process of replacing the first solution at the surface of the sensor element with a second solution, a concentration of the other of the first nucleic acid or the second nucleic acid in the second solution being a second concentration, the second concentration being less than the first concentration; and
a fourth process of detecting the target substance in a state in which the surface of the sensor element is covered with the second solution.


SEQUENCE LISTING (8 sequences)

SEQ ID NO: 1 - DNA, 13 bp, synthetic construct
  source 1..13: mol_type = other DNA
tctctcttggcga

SEQ ID NO: 2 - DNA, 44 bp, synthetic construct
  source 1..44: mol_type = other DNA
ggcgacaaggaaaatccttcaacgaagtgggtcgccaagagaga

SEQ ID NO: 3 - DNA, 12 bp, synthetic construct
  source 1..12: mol_type = other DNA
ctctcttggcga

SEQ ID NO: 4 - DNA, 11 bp, synthetic construct
  source 1..11: mol_type = other DNA
tctcttggcga

SEQ ID NO: 5 - DNA, 10 bp, synthetic construct
  source 1..10: mol_type = other DNA
ctcttggcga

SEQ ID NO: 6 - DNA, 12 bp, synthetic construct
  source 1..12: mol_type = other DNA
tctctcttggcg

SEQ ID NO: 7 - DNA, 11 bp, synthetic construct
  source 1..11: mol_type = other DNA
ctctcttggcg

SEQ ID NO: 8 - DNA, 10 bp, synthetic construct
  source 1..10: mol_type = other DNA
tctcttggcg